(19) United States
(12) Patent Application Publication
(10) Pub. No.: US 2022/0023430 A1
(43) Pub. Date: Jan. 27, 2022

(54) GLYCOPOLYSIALYLATION OF BLINATUMOMAB

(71) Applicant: Lipoxen Technologies Limited, London (GB)

(72) Inventors: Anton Igonin, Gleichen (DE); Dmitry Genkin, St. Petersburg (RU); Curtis Lockshin, Lexington, MA (US)

(73) Assignee: Lipoxen Technologies Limited, London (GB)

(21) Appl. No.: 17/292,911

(22) PCT Filed: Nov. 13, 2019
(86) PCT No.: PCT/EP2019/081231

§ 371 Date: May 11, 2021

Related U.S. Application Data
(60) Provisional application No. 62/760,799, filed on Nov. 13, 2018.

(51) Int. Cl. A61K 47/61; A61K 47/68

(52) U.S. Cl. CPC A61K 47/61; A61K 47/6867; A61K 47/6849

(57) ABSTRACT

A composition comprising a population of polysaccharide-blinatumomab conjugates, wherein the polysaccharide is covalently linked to the blinatumomab. A method of increasing the efficacy of a therapeutic agent in the treatment of B-cell precursor acute lymphoblastic leukemia (ALL), wherein the therapeutic agent is a PSA-drug conjugate, wherein the conjugate comprises PSA covalently linked to blinatumomab, and wherein the PSA of the conjugate binds to DNA and histones of NET extracellular fibrils.

### FIELD OF THE DISCLOSURE

The disclosure relates to polysaccharide-blinatumomab conjugates useful in treating conditions associated with acute lymphoblastic leukemia (ALL), and neutrophil extracellular traps (NETs) and histones. More specifically, the disclosure concerns PSA-blinatumomab conjugates, methods of use, and methods of preparation.

### BACKGROUND OF THE INVENTION

Conjugation of polypeptide drugs such as by PEGylation or polysialylation protects them from degradation in the blood circulation and thus improves their pharmacodynamic and pharmacokinetic profiles (Harris and Chess, Nat Rev Drug Discov. 2003; 2:214-21; S. Jain, D. Hreczuk-Hirst, P. Laing and G. Gregoriadis, Drug Delivery Systems and Sciences, 4 (No 1): 3-9, 2004.). Sialic acids (also called N-acetyl neuraminic acids) and polysialic acids are found widely distributed in animal tissues and to a lesser extent in other species ranging from plants and fungi to yeasts and bacteria, mostly in blinatumomabs and gangliosides. The abbreviation “PSA” used herein refers to the term “polysialic acid”. Similarly, the term “mPSA” used herein refers to the term “modified polysialic acid”.

PSAs consist of polymers (generally homopolymers) of N-acetylneuraminic acid. The secondary amino group normally bears an acetyl group, but it may instead bear a glycolyl group. Possible substituents on the hydroxyl groups include acetyl, lactyl, ethyl, sulfate, and phosphate groups.

PSAs and mPSAs generally comprise linear polymers consisting essentially of N-acetylneuraminic acid moieties linked by 2,8- or 2,9-glycosidic linkages or combinations of these (e.g. alternating 2,8- and 2,9-linkages). In particularly preferred PSAs and mPSAs, the glycosidic linkages are α-2,8. Such PSAs and mPSAs are conveniently derived from colominic acids and are referred to herein as “CAs” and “mCAs”. Typical PSAs and mPSAs comprise at least 2, preferably at least 5, more preferably at least 10 and most preferably at least 20 N-acetylneuraminic acid moieties. Thus, they may comprise from 5 to 500 N-acetylneuraminic acid moieties, preferably from 10 to 300 N-acetylneuraminic acid moieties. PSAs and CAs can be polymers comprising different sugar moieties. They can be copolymers. PSAs and CAs preferably are essentially free of sugar moieties other than N-acetylneuraminic acid. PSAs and CAs preferably comprise at least 90%, more preferably at least 95% and most preferably at least 98% N-acetylneuraminic acid moieties.

Where PSAs and CAs comprise moieties other than N-acetylneuraminic acid (as, for example in mPSAs and mCAs) these are preferably located at one or both of the ends of the polymer chain. Such “other” moieties may, for example, be moieties derived from terminal N-acetylneuraminic acid moieties by oxidation or reduction. For example, WO-A-0187922 describes such mPSAs and mCAs in which the non-reducing terminal N-acetylneuraminic acid unit is converted to an aldehyde group by reaction with sodium periodate. Additionally, WO 2005/016974 describes such mPSAs and mCAs in which the reducing terminal N-acetylneuraminic acid unit is subjected to reduction to reductively open the ring at the reducing terminal N-acetylneuraminic acid unit, whereby a vicinal diol group is formed, followed by oxidation to convert the vicinal diol group to an aldehyde group.

The preparation of conjugates by forming a covalent linkage between the polysaccharide and the therapeutic protein can be carried out by a variety of chemical methods. One approach for coupling PSA to therapeutic proteins is the conjugation of the polymers via the carbohydrate moieties of the protein. Vicinal hydroxyl (OH) groups of carbohydrates in proteins can be easily oxidized with sodium periodate (NalO₄) to form active aldehyde groups (Rothfus and Smith, J Biol Chem 1963; 238:1402-10; van Lenten and Ashwell, J Biol Chem 1971; 246:1889-94). Subsequently the polymer can be coupled to the aldehyde groups of the carbohydrate by use of reagents containing, for example, an active hydrazide group (Wilchek M and Bayer E A, Methods Enzymol 1987; 138:429-42). A more recent technology is the use of reagents containing aminooxy groups which react with aldehydes to form oxime linkages (WO 96/40662, WO2008/025856).

Blinatumomab (Blincyto®) is a monoclonal antibody used in pediatric and adult patients for the following indications:
a) Acute lymphoblastic leukemia (B-cell precursor), minimal residual disease (MRD)-positive (≥0.1%); and
b) Acute lymphoblastic leukemia (B-cell precursor), relapsed/refractory.

Blinatumomab is a bi-specific anti-CD19/CD3 (BiTE®) antibody. However, toxicity is significant with blinatumomab. In this aspect, 15% of patients in the pivotal trials of Blincyto® in ALL and 7% after in MDR in ALL developed cytokine release syndrome (CRS). The non-antigen specific toxicity is a result of a high-level systemic inflammatory response syndrome triggered by Blincyto®. The hallmark of CRS is a supraphysiological level of inflammatory cytokines IFNγ, TNFα, IL-1β, IL2, IL4, IL-6, IL-8, IL-10, IL-12, etc. This inflammatory response affords arterial hypotension and acute respiratory distress syndrome (ARDS) as the prominent features of an acute organ damage in terms of severe CRS. According to Lee (Lee et al., Blood; 2014; 124(2):188-195) the goal of CRS treatment is to prevent life-threatening toxicity while maximizing the potential for antitumor effects. Treatment of severe CRS includes anti-human IL-6R Ab and steroids. Extracellular histones are involved in this cytokine release of CRS.

According to Galuska (Galuska et al.; Frontiers in Immunology; September 2017; Vol. 8; Art. 1229), neutrophils are involved in numerous immunological events. One mechanism of neu-trophils to combat pathogens is the formation of NETs. Thereby, neutrophils use DNA fibers to form a meshwork of DNA and histones as well as several antimicrobial components to trap and kill invaders. However, the formation of NETs can lead to pathological conditions triggering among other things (e.g., sepsis or acute lung failure), which is mainly a consequence of the cytotoxic characteristics of accumulated extracellular histones. Histones play an active role in the ALL pathogenesis promoting endothelial adhesion of leukemic cells and their resistance to chemotherapeutic agents. After incubation with PSA such protective and pro-adhesive histones activity was shown to be significantly diminished (Yoo et al., PLoS One., Oct. 5, 2016; 11(10):e0163982)

Elimination of CRS with an increase of T/2 may lead to optimization of drug delivery (iv instead of continuous infusion) with subsequent improvement of compliance and quality of life. Elimination of CRS would allow to deliver the full dose of Blincyto. In those subjects who developed this adverse effect there remains a need to develop materials and methods for conjugating polysaccharides to blinatumomab that improve the compound's pharmacodynamic and/or pharmacokinetic properties and/or minimizing or eliminating CRS and/or minimizing or eliminating histone-mediated organ damage and symptoms.

### SUMMARY

Aspects of the present disclosure teach certain benefits in construction and use which give rise to the exemplary advantages described below.

In one aspect, disclosed herein is a composition comprising a population of polysaccharide-blinatumomab conjugates, wherein the polysaccharide is covalently linked to the blinatumomab.

In another aspect, disclosed herein is a composition as disclosed herein, wherein the composition is a pharmaceutical composition and comprises one or more pharmaceutically acceptable excipients.

In another aspect, disclosed herein is a method of increasing the efficacy of a therapeutic agent in the treatment of B-cell precursor acute lymphoblastic leukemia (ALL), wherein the therapeutic agent is a PSA-drug conjugate, wherein the conjugate comprises PSA covalently linked to blinatumomab, and wherein the PSA of the conjugate binds to DNA and histones of NET extracellular fibrils.

In another aspect, disclosed herein is a method of treating B-cell precursor acute lymphoblastic leukemia (ALL), wherein the method comprises administering an effective amount of a PSA-drug conjugate to a patient in need thereof, wherein the PSA-drug conjugate comprises PSA covalently linked to blinatumomab, and wherein PSA of the conjugate binds to DNA and histones of NET extracellular fibrils.

In another aspect, disclosed herein is a composition comprising a population of polysaccharides and blinatumomab.

In another aspect, disclosed herein is method of treating B-cell precursor acute lymphoblastic leukemia (ALL), wherein the method comprises administering effective amounts of a PSA and blinatumomab to a patient in need thereof, wherein the PSA binds to DNA and histones of NET extracellular fibrils.

In yet another aspect, disclosed herein is a method for producing a polysaccharide derivative of blinatumomab, wherein an anionic polysaccharide comprising 2-200 saccharide units, is chemically reacted with the blinatumomab.

### BRIEF DESCRIPTION OF THE DRAWINGS

The accompanying drawings illustrate aspects of the present disclosure.

FIG. 1a is a reaction scheme showing the prior art activation of the non-reducing sialic acid terminal unit.

FIG. 1b is a reaction scheme showing the prior art reductive amination of the aldehyde moiety of the product of reaction scheme 1a using a protein-amine moiety.

FIG. 2a is a schematic diagram showing the potential side-reactions taking place in the reaction of FIG. 1b involving the reducing terminal.

FIG. 2b represents schematically the potential by-products of the side reactions of FIG. 2a.

FIG. 3 is a reaction scheme showing the tautomerism between the ketal and ring-closed forms of the reducing terminal sialic acid unit of a PSA.

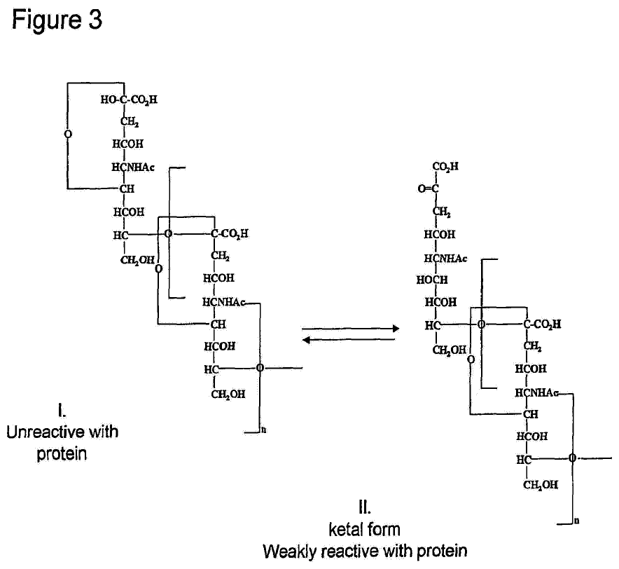

FIG. 4a is a reaction scheme showing the preferred oxidation-reduction oxidation reactions of PSA.

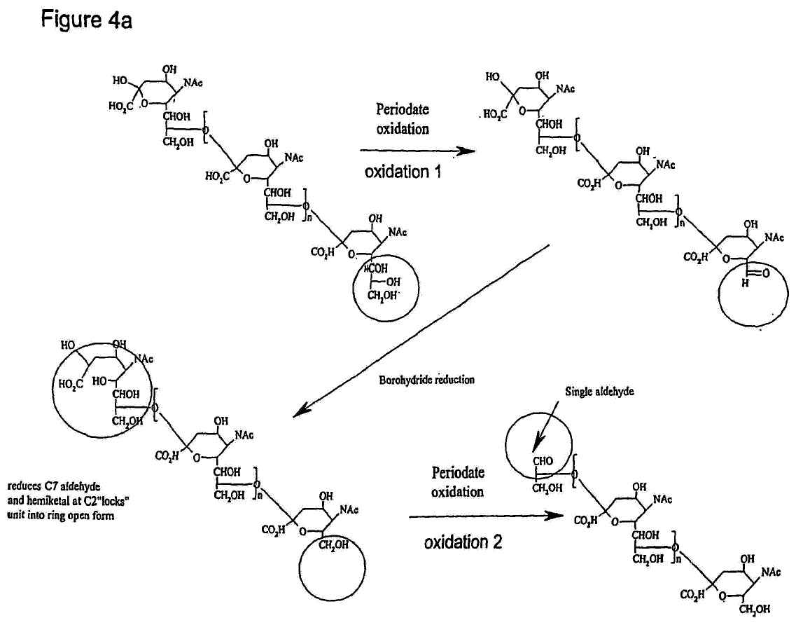

FIG. 4b gives suitable conditions for the steps of the scheme of FIG. 4 and explains abbreviations used for the starting materials, intermediates and end products.

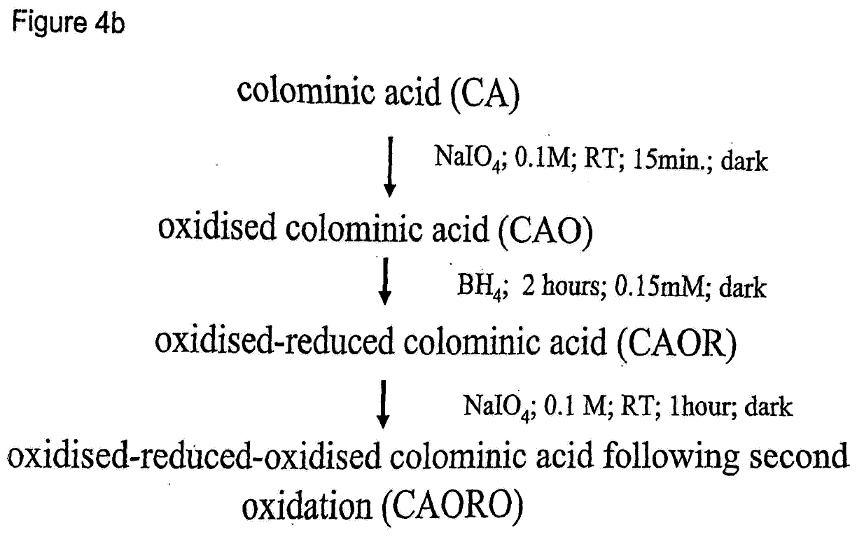

FIG. 5 is a schematic diagram similar to FIG. 2b but shows the products of the reaction of FIG. 4.

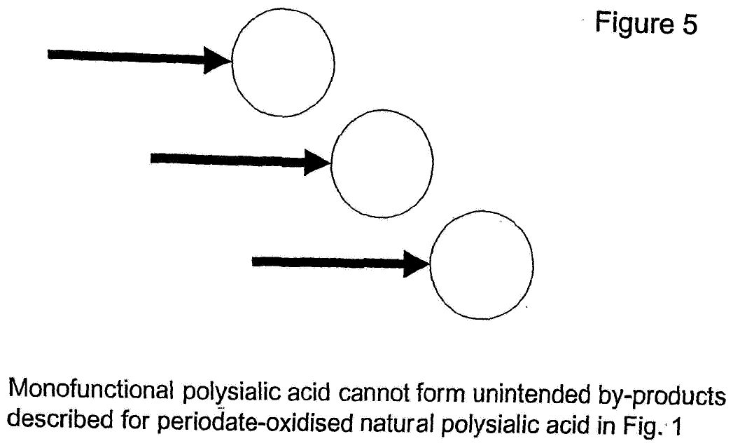

The above described drawing figures illustrate aspects of the disclosure in at least one of its exemplary embodiments, which are further defined in detail in the following description. Features, elements, and aspects of the disclosure that are referenced by the same numerals in different figures represent the same, equivalent, or similar features, elements, or aspects, in accordance with one or more embodiments.

### DETAILED DESCRIPTION

Disclosed herein are polysaccharide-blinatumomab conjugates that binds to CD3 of T-cells and/or CD19 of B-cells. Polysaccharide-blinatumomab conjugates can be used to treat B-cell precursor acute lymphoblastic leukemia (ALL), while minimizing or eliminating CRS and/or minimizing or eliminating histone-mediated organ damage and symptoms. Exemplary compositions described herein include formulations of PSA-blinatumomab conjugates for the treatment of B-cell precursor acute lymphoblastic leukemia (ALL) and minimizing or eliminating CRS and histone-mediated organ damage and symptoms.

In one aspect, disclosed herein is a composition comprising a population of polysaccharide-blinatumomab conjugates, wherein the polysaccharide is covalently linked to the blinatumomab.

In the disclosure the polysaccharide may be a naturally occurring polysaccharide, or a derivative of a naturally occurring polysaccharide, for instance, a polysaccharide which has been derivatized by a reaction of one or more active groups on the saccharide residues, or which has been covalently linked to a derivatizing group at the end of the polysaccharide chain.

The polysaccharide may be linked to the blinatumomab via either its reducing or non-reducing terminal unit.

In some embodiments, the polysaccharide is polysialic acid, heparin, hyaluronic acid or chondroitin sulphate. In particular embodiments, the polysaccharide is polysialic acid (PSA) or a modified PSA (mPSA). In some embodiments, the polysaccharide is polysialic acid consisting substantially only of sialic acid units. The PSA or mPSA may have a molecular weight range of 350 Da to 120,000 Da, 500 Da to 100,000 Da, 1000 Da to 80,000 Da, 1500 Da to 60,000 Da, 2,000 Da to 45,000 Da or 3,000 Da to 35,000 Da. The PSA or mPSA may be colominic acid or modified colominic acid. In another embodiment of the invention, the PSA or mPSA is comprised of about 2-500 or 10-300 sialic acid units. In some embodiments, the polysaccharides comprise 10-80 sialic acid units. In some embodiments, the polysaccharide comprises between 2 and 125 saccharide units. Preferably the polysaccharide starting material has at least 2, more preferably at least 5, more preferably at least 10, for instance at least 50, saccharide units. For instance, a polysaccharide may comprise at least 5 sialic acid units.

In the disclosure herein, the polysaccharide starting material may comprise units other than sialic acid in the molecule. For instance, sialic acid units may alternate with other saccharide units. Preferably, however, the polysaccharide consists substantially only of units of sialic acid. Preferably these are joined 2-8 and/or 2-9. In some embodiments, the polydispersity of the anionic polysaccharide is less than 1.3.

The polysialic acid may be derived from any source preferably a natural source such as a bacterial source, e.g. E. coli K1 or K92, group B meningococci, or even cow's milk or N-CAM the sialic acid polymer may be a heteropolymeric polymer such as group 135 or group V of N. meningitidis. The polysialic acid may be in the form of a salt or the free acid. It may be in a hydrolyzed form, such that the molecular weight has been reduced following recovery from a bacterial source. The polysialic acid may be material having a wide spread of molecular weights such as having a polydispersity of more than 1.3, for instance as much as 2 or more. Preferably the polydispersity of molecular weight is less than 1.2, for instance as low as 1.01.

A population of polysialic acids having a wide molecular weight distribution may be fractionated into fractions with lower polydispersities, i.e. into fractions with differing average molecular weights. Fractionation is preferably anion exchange chromatography, using for elution a suitable basic buffer. We have found a suitable anion exchange medium i) a preparative medium such as a strong ion-exchange material based on activated agarose, having quaternary ammonium ion pendant groups (ie strong base). The elution buffer is non-reactive and is preferably volatile so that the desired product may be recovered from the base in each fraction by evaporation. Suitable examples are amines, such as triethanolamine. Recovery may be by freeze-drying for instance. The fractionation method is suitable for a polysialic acid starting material as well as to the derivatives. The technique may thus be applied before or after the essential process steps of this invention.

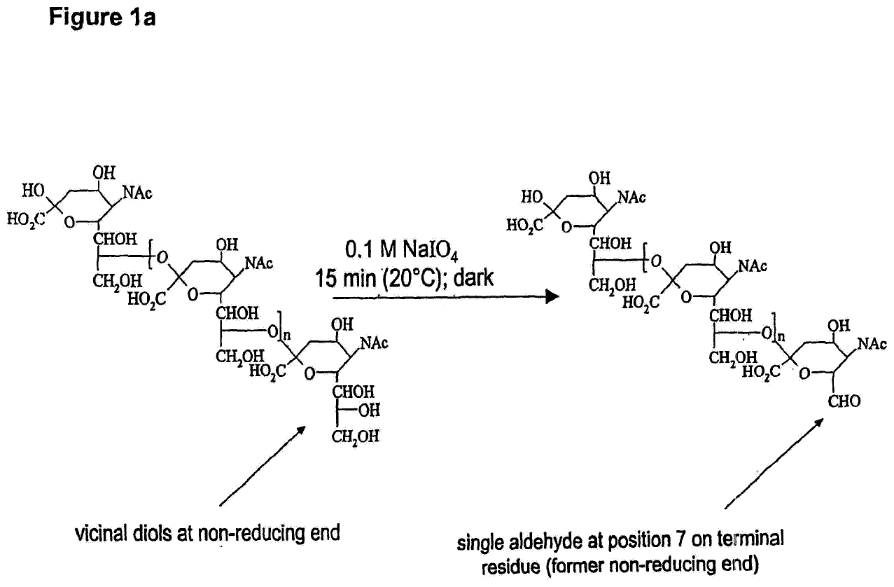

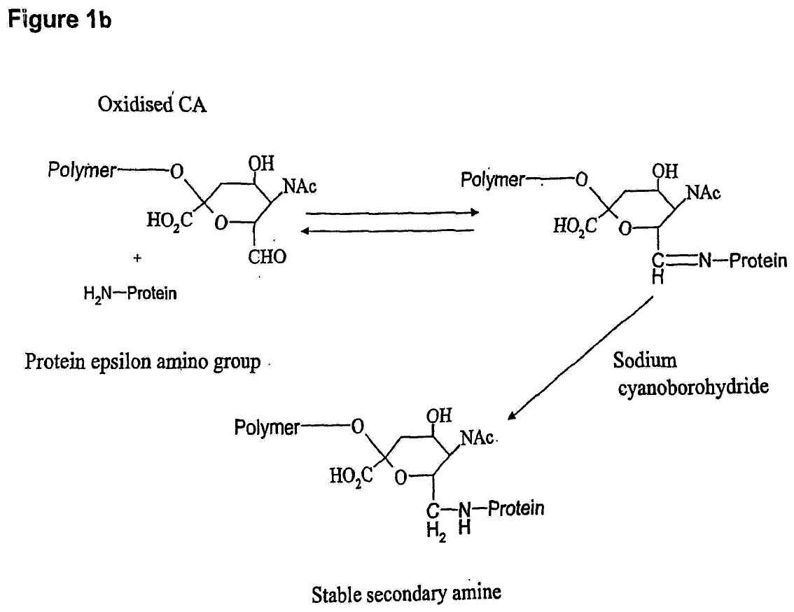

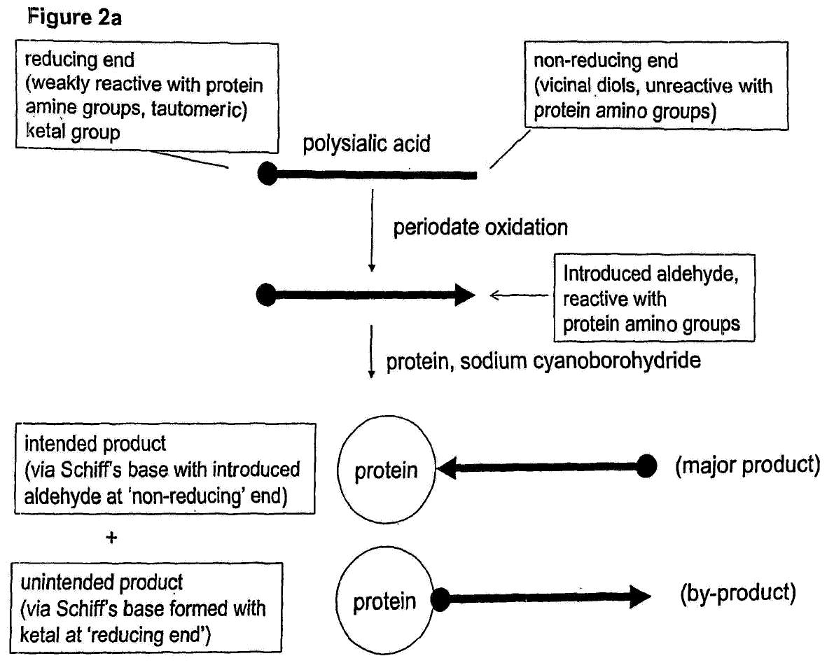

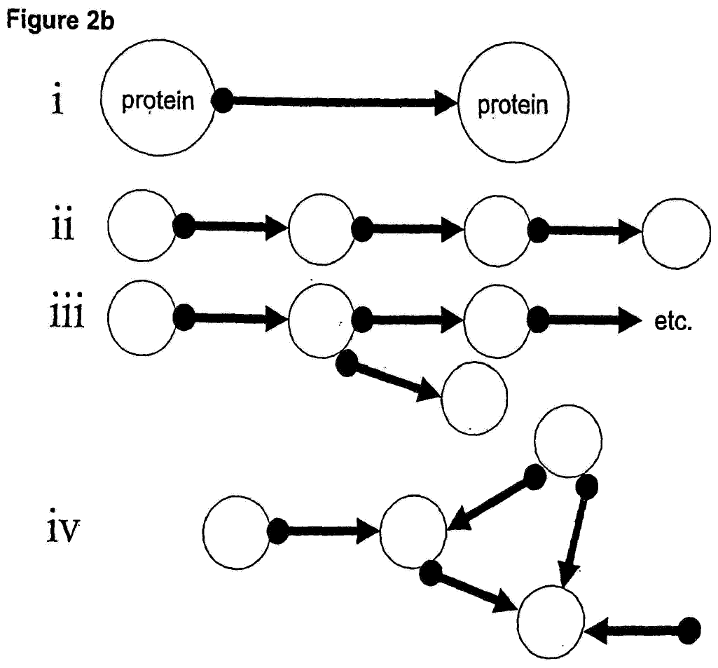

The polysaccharide may be linked to the blinatumomab monoclonal antibody directly, i.e. as shown in FIGS. 1 and 2, or via a linker. Suitable linkers are derived from N-maleimide, vinylsulphone, N-iodoacetamide, orthopyridyl or N-hydroxysuccinimide-containing reagents. The linker may also be biostable or biodegradable and comprise, for instance, a polypeptide or a synthetic oligomer. The linker may be derived from a bifunctional moiety, as further described in WO 2005/016973. A suitable bifunctional reagent is, for instance, Bis-NHS. The reagent may have general formula Z—R¹—Z wherein each Z is a functional group and may be the same or different and R¹ is a bifunctional organic radical. Preferably, R¹ is selected from the group consisting of alkanediyl, arylene, alkarylene, heteroarylene and alkylheteroarylene, any of which may substituted and/or interrupted by carbonyl, ester, sulfide, ether, amide and/or amine linkages. Particularly preferred is C₃-C₆ alkanediyl. Most preferably, R¹ corresponds to the appropriate portion of the suitable bifunctional reagent

In some embodiments, the polysaccharide derivatives are compounds of Formula (I):

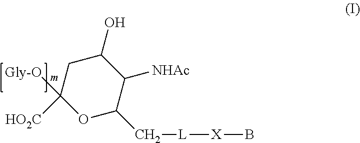

wherein
m is at least one;
XB is derived from B—XH which is blinatumomab, wherein XH is NH₂ or SH;
L is a bond, a linking group, or comprises a polypeptide or a synthetic oligomer;
GlyO is an anionic saccharide unit,
wherein the linking group, if present, is of general formula —Y—C(O)—R¹—C(O)—;
wherein Y is NR² or NR²—NR²; R¹ is a difunctional organic radical selected from the group consisting of alkanediyl, arylene, alkarylene, heteroarylene and alkylheteroarylene, any of which may substituted and/or interrupted by carbonyl, ester, sulfide, ether, amide and/or amine linkages; and
R² is H or C₁₋₆ alkyl.

In some embodiments, L is a bond or is a group

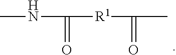

The pharmaceutical composition may be in the form of an aqueous suspension. Aqueous suspensions contain the novel compounds in admixture with excipients suitable for the manufacture of aqueous suspensions. The pharmaceutical compositions may be in the form of a sterile injectable aqueous or homogeneous suspension. This suspension may be formulated according to the known art using suitable dispersing or wetting agents and suspending agents.

Pharmaceutical compositions may be administered orally, intravenously, intraperitoneally, intramuscularly, subcutaneously, intranasally, intradermally, topically or intratracheally for human or veterinary use.

The compositions may further comprise a formulation additive. By formulation additive we mean an excipient which is capable of stabilizing the insulin either internally or externally, as described in Wang et al (1999). The excipient may be a stabilizer, a solubilizer or a metal ion. Suitable examples of formulation additives include one or more buffers, stabilizers, surfactants, salts, polymers, metal ions, sugars, polyols or amino acids. These may be used alone or in combination.

Stabilizers typically act by destabilization of the denatured state of a protein leading to increased Gibbs free energy change for unfolding of the protein. The stabilizer is preferably a sugar or a polyol, for example sucrose, sorbitol, trehalose, glycerol, mannitol, lactose and ethylene glycol. A stabilizing buffer is sodium phosphate.

The solubilizer is preferably a surfactant, preferably a non-ionic surfactant. Suitable examples include Tween 80, Tween 20, Tween 40, Pluoronic® F68, Brij 35 and Triton X100.

The metal ion is preferably divalent. Suitable metal ions include Zn2+, Ni²⁺, Co²⁺, Sr²⁺, Cu²⁺, Ca²⁺, Mg²⁺ and Fe²⁺.

The formulation additive may also be a polymer selected from PSA, PEG or hydroxy-beta-cyclodextrin. Preservatives such as m-Cresol may also be used. Suitable amino acids and amino acid derivatives for use as the formulation additive include histidine, glycine, other similar amino acids and sodium aspartate.

The phase “chemically reacted substantially only at the N-terminal amine” means that in a population of derivatives at least 85%, more preferably at least 90%, most preferably at least 95% of the protein is derivatized only at its N-terminal amine.

In another aspect, disclosed herein is a composition comprising a population of polysaccharides and blinatumomab. In some embodiments, the polysaccharide comprises between 2 and 125 saccharide units. In some embodiments, the polysaccharide is polysialic acid (PSA), heparin, hyaluronic acid or chondroitin sulphate. In some embodiments, the polysaccharide is PSA consisting substantially only of sialic acid units. In some embodiments, polysaccharides comprise 10-80 sialic acid units.

In another aspect, disclosed herein is a composition as disclosed herein, wherein the composition is a pharmaceutical composition and comprises one or more pharmaceutically acceptable excipients.

NETs have been shown to contribute to autoimmune diseases, cancer, myocardial ischemia/reperfusion injury, deep vein thrombosis, stroke, pancreatitis, liver injury, kidney failure, coagulation, retinal detachment, and apoptosis. Therefore, NETosis is key to the development of novel therapeutic interventions for a wide array of clinical situations.

In another aspect, disclosed herein is a method of increasing the efficacy of a therapeutic agent in the treatment of B-cell precursor acute lymphoblastic leukemia (ALL), wherein the therapeutic agent is a PSA-drug conjugate, wherein the conjugate comprises PSA covalently linked to blinatumomab, and wherein the PSA of the conjugate binds to DNA and histones of NET extracellular fibrils.

In another aspect, disclosed herein is a method of treating B-cell precursor acute lymphoblastic leukemia (ALL), wherein the method comprises administering an effective amount of a PSA-drug conjugate to a patient in need thereof, wherein the PSA-drug conjugate comprises PSA covalently linked to blinatumomab, and wherein PSA of the conjugate binds to DNA and histones of NET extracellular fibrils.

Flow cytometry (FLOW)-based assays are used to identify and quantify NETs using antibodies against key NETs constituents as DNA and histones. This method is applicable for the assessment of induced NETs in vitro, or detection of NETosis in vivo in blood samples.

In another aspect, disclosed herein is a method for determining a dosage amount to administer to a subject a PSA or a PSA-drug conjugate for treatment of histone-mediated cytotoxicity by determining a histone binding response in blood in the subject, the method comprising:

(i) collecting a first blood sample from the subject;

(ii) quantifying histones of the first blood sample of step (i);

(iii) administering to the subject a first amount of a PSA or a PSA-drug conjugate;

(iv) collecting a second blood sample from the subject;

(v) quantifying histones of the second blood sample of step (iv); and

(vi) determining whether to increase or reduce the PSA or the PSA-drug dosage administered based upon the pharmacodynamic effect as determined by a degree of the histone binding observed based upon the quantitative determination of steps (ii) and (v).

In some embodiments, ALL of a subject is in remission but still have minimal residual disease (MRD), Philadelphia chromosome (Ph)-negative relapsed or refractory positive B-cell precursor ALL, and/or Philadelphia chromosome (Ph)-positive relapsed or refractory positive B-cell precursor ALL.

One mechanism that neutrophils employ to defend against pathogens is the formation of NETs. With this mechanism, neutrophils form a meshwork of DNA and histones as well as several antimicrobial components to trap and kill invaders. However, the formation of NETs leads to pathological conditions, which is mainly a consequence of the cytotoxic characteristics of accumulated extracellular histones.

In some embodiments, the pharmacokinetic properties of the conjugate of blinatumomab are enhanced as compared to the blinatumomab alone

PSA is an antagonist of the cytotoxic properties of extracellular histones. The PSA chains disclosed herein administered alone or as a conjugate effectively neutralize histone-mediated cytotoxicity and initiate binding to NET filaments. Therefore, PSA and PSA-conjugates disclosed herein are useful agents to reduce histone-mediated cytotoxicity but also an anchor to accumulate PSA and PSA-conjugates in areas of NET formation. In some embodiments, the PSA of the conjugate can direct the therapeutic protein to Neutrophil Extracellular Traps (NETs) via an affinity of PSA to histones in NETs.

In some embodiments, the PSA of the conjugate binds to histones in the plasma but not in NETs. In some embodiments, the PSA binds to histones of histone families H1/H5, H2A, H2B, H3, H4, or combinations thereof.

In some embodiments, the PSA of the conjugate binds to histones in plasma but not in NETs and the blinatumomab of the conjugate binds to CD3 of T-cells and/or CD19 of B-cells.

In some embodiments, the PSA of the conjugate is released from the conjugate binds to DNA and histones of NET extracellular fibrils and/or histones in plasma.

In some embodiments, administration of the conjugate reduces blinatumomab toxicity. In some embodiments, cytokine release syndrome (CRS) resulting from blinatumomab toxicity is prevented or symptoms of CRS reduced. In some embodiments, the PSA of the conjugate reduces histone-mediated organ damage. In some embodiments, the cytotoxicity of the blinatumomab of the conjugate is enhanced via down-regulation of leukemic cells adhesion to endothelium.

In some embodiments, the conjugate is administered orally or via subcutaneous injection.

In some embodiments, the PSA of the conjugate or PSA released from the conjugate reduces cancer metastasis. In some embodiments, the PSA of the conjugate or PSA released from the conjugate reduces NET-mediated cancer metastasis.

In some embodiments, the PSA of the conjugate or PSA released from the conjugate reduces NET-mediated inflammation.

In some embodiments, the PSA of the conjugate or PSA released from the conjugate decreases blood clearance of interferon-γ and increases interferon-γ activity.

In some embodiments, the polysaccharide is PSA, and wherein the PSA of the conjugate prevents degradation of blinatumomab from serum proteases.

In some embodiments, the conjugate retains the biological activity of blinatumomab in the presence of anti-CD3 antibodies and/or anti-CD19 antibodies. In some embodiments, the conjugate retains the biological activity of blinatumomab in the presence of anti-blinatumomab antibodies. In some embodiments, the pharmacokinetic properties of the conjugate of blinatumomab are enhanced as compared to the blinatumomab alone. In some embodiments, the pharmacokinetic properties of the conjugate of blinatumomab are maintained as compared to the blinatumomab alone. In some embodiments, the pharmacokinetic properties of the conjugate of blinatumomab are maintained as compared to the blinatumomab alone.

In some embodiments, the conjugate of blinatumomab has at least at least a 2-fold increase in beta-phase blood clearance rate as compared to blinatumomab alone. In some embodiments, the conjugate of blinatumomab has at least at least a 1.8-fold increase in tumor exposure as compared to blinatumomab alone.

In another aspect, disclosed herein is a method of treating B-cell precursor acute lymphoblastic leukemia (ALL), wherein the method comprises administering effective amounts of a PSA and blinatumomab to a patient in need thereof, wherein the PSA binds to DNA and histones of NET extracellular fibrils.

In some embodiments, the pharmacokinetic properties of blinatumomab administered with PSA are enhanced as compared to blinatumomab administered alone.

In some embodiments, the PSA can direct the therapeutic protein to Neutrophil Extracellular Traps (NETs) via an affinity of PSA to histones in NETs. In some embodiments, the PSA binds to histones in the plasma but not in NETs. In some embodiments, the PSA binds to histones of histone families H1/H5, H2A, H2B, H3, H4, or combinations thereof. In some embodiments, the PSA binds to histones in plasma but not in NETs and the blinatumomab binds to CD3 of T-cells and/or CD19 of B-cells.

In some embodiments, administration of the PSA reduces blinatumomab toxicity. In some embodiments, cytokine release syndrome (CRS) resulting from blinatumomab toxicity is prevented or symptoms of CRS reduced. In some embodiments, the PSA reduces histone-mediated organ damage. In some embodiments, the cytotoxicity of the blinatumomab is enhanced via PSA down-regulation of leukemic cells adhesion to endothelium.

In some embodiments, the PSA and blinatumomab are independently administered orally or via subcutaneous injection.

In some embodiments, the PSA reduces NET-mediated cancer metastasis.

In some embodiments, the PSA reduces NET-mediated inflammation.

In some embodiments, the PSA decreases blood clearance of interferon-γ and increases interferon-γ activity.

In some embodiments, the PSA prevents degradation of blinatumomab from serum proteases.

In some embodiments, the blinatumomab and PSA retains the biological activity of blinatumomab in the presence of anti-CD3 antibodies and/or anti-CD19 antibodies. In some embodiments, the blinatumomab and PSA retains the biological activity of blinatumomab in the presence of anti-blinatumomab antibodies. In some embodiments, the pharmacokinetic properties of blinatumomab administered with PSA are enhanced as compared to the blinatumomab alone.

In some embodiments, the pharmacokinetic properties of blinatumomab administered with PSA are maintained as compared to the blinatumomab alone.

In some embodiments, the blinatumomab administered with PSA has at least a 2-fold increase in beta-phase blood clearance rate as compared to blinatumomab alone. In some embodiments, the blinatumomab administered with PSA has at least a 1.8-fold increase in tumor exposure as compared to blinatumomab alone.

In yet another aspect, disclosed herein is a method for producing a polysaccharide derivative of blinatumomab, wherein an anionic polysaccharide comprising 2-200 saccharide units, is chemically reacted with the blinatumomab. In some embodiments, the blinatumomab is derivatized by the polysaccharide at the reducing terminal unit of the polysaccharide.

Disclosed herein is a method for conjugation of polysaccharides to proteins whereby the high reactivity of the N-terminal of the protein can be utilized and which avoids the product complexity obtained using the established method (FIGS. 1 and 2) of reductive amination of proteins with periodate oxidized natural colominic acid.

The polysaccharide may also react with a modified form of blinatumomab. For instance, one or more groups on the blinatumomab may have undergone a chemical transformation, for instance, by reduction or oxidation. A reactive carbonyl may be generated in the place of the terminal amino group of blinatumomab using oxidation conditions, for instance.

Suitable polysaccharides for use in the method of this invention are as described previously for the novel compositions.

The compounds of the invention may be manufactured by any of the suitable methods described in the prior art. For example, a typical method is described to our previous patent application WO 92/22331.

Typically, the anionic polysaccharide has been activated before derivatization to insulin. It may, for instance, have a reactive aldehyde group and the derivatization reaction may be carried out under reducing conditions. In some embodiments, the anionic polysaccharide has a reactive aldehyde group which reacts with the blinatumomab and the derivatization reaction is carried out under reducing conditions. The reactive aldehyde group may be produced by controlled oxidation of a hydroxyl group of the polysaccharide. Most preferably this reactive aldehyde is generated in a preliminary step, in which the polysaccharide is reacted under controlled oxidation conditions, for instance using sodium periodate, in aqueous solution. Preferably the oxidation is a chemical oxidation, although enzymes which are capable of carrying out this step may also be used. The reactive aldehyde group may be at the non-reducing end or reducing end of the polysaccharide. In some embodiments, the reactive aldehyde group is at the non-reducing end of the polysaccharide. In some embodiments, the reactive aldehyde is at the reducing end of the polysaccharide and the non-reducing end has been passivated such that it does not react with the blinatumomab. The blinatumomab, typically the N-terminus, may then react with the reactive aldehyde group to produce an adduct which, when reduced, produces the N-terminal derivative of blinatumomab.

The activation of the polysaccharide should preferably be carried out under conditions such that there is substantially no mid-chain cleavage of the backbone of the polysaccharide, that is substantially no molecular weight reduction. The oxidant is suitably perrhuthenate, or, preferably, periodate. Oxidation may be carried out with periodate at a concentration in the range 1 mM to 1M, at a pH in the range 3 to 10, a temperature in the range 0 to 60° C. for a time in the range 1 min to 48 hours.

Suitable reduction conditions for the derivatization reaction may utilize hydrogen with catalysts or, preferably hydrides, such as borohydrides. These may be immobilized such as Amberlite™ supported borohydride. Preferably alkali metal hydrides such as sodium borohydride is used as the reducing agent, at a concentration in the range 1 μM to 0.1 M, a pH in the range 4 to 10, a temperature in the range 0 to 60° C. and a period in the range 1 min to 72 hours. The reaction conditions are selected such that pendant carboxyl groups on the starting material are not reduced. Other suitable reducing agents are cyanoborohydride under acidic conditions, e.g. polymer supported cyanoborohydride or alkali metal cyanoborohydride, L-ascorbic acid, sodium metabisulphite, L-selectride, triacetoxyborohydride etc.

Other activated derivatives of polysaccharides may have utility in the present disclosure, including those with pendant functional groups such as NHS, as described in our earlier patent application WO 06/00540.

In one embodiment, the reactive aldehyde is at the reducing end of the polysaccharide and the non-reducing end has been passivated such that it does not react with pendant groups on the blinatumomab.

The reactivity of the reducing end of colominic acid, though weak towards protein targets, is sufficient to be troublesome in the manufacture of chemically defined conjugates.

The polysaccharide may be derivatized before it reacts with blinatumomab. For instance, the polysaccharide may react with a bifunctional reagent.

The polysaccharide may be subjected to a preliminary reaction step, in which a group selected from a primary amine group, a secondary amine group and a hydrazine is formed on the terminal saccharide, which is preferably sialic acid, followed by a reaction step in which this is reacted with a bifunctional reagent to form a reaction-intermediate, as further described in WO 2006/016168. The intermediate may then react with the blinatumomab. The bifunctional reagent may have general formula Z—R¹—Z, as defined previously.

We have found that certain reaction conditions promote selective derivatization at the N-terminal of the blinatumomab. To promote selective reaction at the N-terminal, the derivatization reaction should be carried out in a first aqueous solution of acidic pH, and the resultant polysaccharide derivative should then be purified in a second aqueous solution of higher pH than the first aqueous solution. By acidic pH we mean a pH less than 7. Typically, the pH of the first aqueous solution is in the range 4.0-6.5, preferably 4.0-6.0 and the pH of the second aqueous solution is in the range of 6.5-9.0, preferably 6.5-8.5 or 6.5-8.0. The low pH of the derivatization reaction promotes selective derivatization at the N-terminus of the protein rather than at any mid-chain sites.

Furthermore, we have found that the use of certain formulation additives promotes the formation of a selective, stable, polysaccharide blinatumomab-derivative. The formulation additive may be selected from one or more buffers, stabilizers, surfactants, salts, polymers, metal ions, sugars, polyols or amino acids. These may be added to the reaction medium, or alternatively may be added to the final product composition, as a stabilizer.

In one embodiment of this invention, the formulation additive is sorbitol, trehalose or sucrose. In a different embodiment, the formulation additive is a non-ionic surfactant. The formulation additive may alternatively be a polymer selected from PSA, PEG or hydroxy-beta-cyclodextrin. In a different embodiment the formulation additive is a divalent metal ion. Preferred divalent metal ions include Zn²⁺, Ni²⁺, Co²⁺, Sr²⁺ or Fe²⁺.

The formulation additive may be a buffer. Preferably when the formulation additive is a buffer, it is sodium phosphate or sodium acetate.

The purification of the polysaccharide derivative in the method of the present invention may be carried out using a variety of methods known in the art. Examples of suitable purification methods include HIC (hydrophobic interaction chromatography), SEC (size exclusion chromatography), HPLC (high performance liquid chromatography), and IEC (ion exchange chromatography).

A population of polysialic acids having a wide molecular weight distribution may be fractionated into fractions with lower polydispersities, i.e. into fractions with differing average molecular weights. Fractionation is preferably performed by anion exchange chromatography, using for elution a suitable basic buffer, as described in our earlier patent applications WO 2005/016794 and WO 2005/03149. The fractionation method is suitable for a polysaccharide starting material as well as to the derivatives. The technique may thus be applied before or after the essential process steps of this invention. Preferably, the resultant polysaccharide derivative of insulin has a polydispersity of less than 1.1.

The derivatization of blinatumomab in accordance with this disclosure, results in increased half-life, improved stability, reduced immunogenicity, and/or control of solubility and hence bioavailability and the pharmacokinetic properties of blinatumomab. The new method is of particular value for creation of a monopolysialylated-blinatumomab conjugates.

In some embodiments, the anionic polysaccharide has a reactive aldehyde group which is converted in a preliminary reaction step into an amine, which is then reacted with a bifunctional reagent comprising at least one functional group selected from N-maleimide, vinyl sulphone, N-iodoacetamide, orthopyridyl group or N-hydroxysuccinimide, to form a reaction intermediate, wherein the reaction intermediate is reacted with the blinatumomab.

In some embodiments, the anionic polysaccharide or reaction intermediate reacts with a terminal amine group of the blinatumomab in a first aqueous solution of acidic pH; and the resultant polysaccharide derivative is purified in a second aqueous solution of higher pH than the first aqueous solution. In some embodiments, the polysaccharide reacts with an amine group of the blinatumomab. In some embodiments, the amine is a terminal amine group.

In some embodiments, the pH of the first aqueous solution is in the range 4.0-6.0 and the pH of the second aqueous solution is in the range 6.5-9.0.

In some embodiments, the method carried out in the presence of a formulation additive. In some embodiments, the formulation additive is selected from one or more buffers, stabilizers, surfactants, salts, polymers, metal ions, sugars, polyols, amino acids, or a combination thereof. In some embodiments, the formulation additive is sorbitol, trehalose, sucrose, or a combination thereof. In some embodiments, the formulation additive is a non-ionic surfactant. In some embodiments, the formulation additive is a polymer selected from PSA, PEG or hydroxy-beta-cyclodextrin. In some embodiments, the formulation additive is a divalent metal ion, preferably Zn²⁺, Ni²⁺, Co²⁺, Sr²⁺, Fe²⁺, Mg²⁺, Ca²⁺, or a combination thereof. In some embodiments, the formulation additive is a buffer and the buffer is sodium phosphate.

In further embodiments, there is provided a method of conjugating a polysaccharide to an oxidized carbohydrate moiety of blinatumomab, comprising contacting the oxidized moiety of blinatumomab with the polysaccharide under conditions that allow conjugation, wherein the polysaccharide contains an aminooxy group and an oxime linkage is formed between the oxidized carbohydrate moiety of blinatumomab and the aminooxy group on the water soluble polysaccharide, or wherein the polysaccharide contains a hydrazide group and a hydrazone linkage is formed between the oxidized carbohydrate moiety of blinatumomab and the hydrazide group on the polysaccharide.

The oxidized moiety of blinatumomab may be oxidized using a sugar-specific oxidizing enzyme (e.g. galactose or glucose oxidase) or by incubation with a buffer comprising an oxidizing agent selected from sodium periodate (NalO₄), lead tetraacetate (Pb(OAc)₄) and potassium perruthenate (KRuO₄).

The oxidized carbohydrate moiety of blinatumomab may be oxidized at a sialic acid, mannose, galactose or glucose residue.

In yet another embodiment, the aforementioned method is provided wherein the oxidizing agent is sodium periodate (NalO₄).

The method of the invention may comprise oxidizing the polysaccharide to form an aldehyde group on a terminal sialic acid unit of the polysaccharide and reacting the oxidized polysaccharide with an aminooxy linker.

In yet another embodiment of the disclosure, the aforementioned method is provided wherein the polysaccharide is prepared by reacting an activated aminooxy linker with oxidized polysaccharide wherein the linker is a homo-bifunctional or heterobifunctional linker. The homo-bifunctional linker can have the general formula NH₂[OCH₂CH₂]ₙONH₂, wherein n=1-50, preferably 1-11, more preferably 1-6. The linker may specifically be selected from:

a 3-oxa-pentane-1,5-dioxyamine linker of the formula:

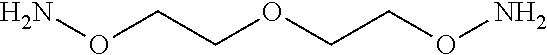

and

a 3,6,9-trioxa-undecane-1,11-dioxyamine linker of the formula:

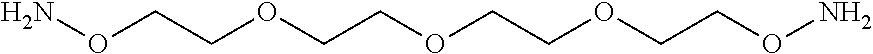 [SMILES: NOCCOCCOCCOCCON]

PSA or mPSA may be oxidized by incubation with an oxidizing agent to form a terminal aldehyde group at the non-reducing end of the PSA.

The method may comprise oxidizing the polysaccharide to form an aldehyde group on a terminal unit of the polysaccharide, e.g. a terminal sialic acid unit of the PSA or mPSA and reacting the oxidized polysaccharide with an aminooxy linker. In still another embodiment, an aforementioned method is provided wherein the aminooxy linker is 3-oxa-pentane-1,5-dioxyamine. In a related embodiment, the oxidizing agent is NalO₄.

In another embodiment of the invention, the aforementioned method is provided wherein the contacting of the oxidized carbohydrate moiety of blinatumomab with the activated polysaccharide occurs in a buffer comprising a nucleophilic catalyst selected from the group consisting of aniline and aniline derivatives.

A hydrazide group may be formed on the polysaccharide by reacting oxidized polysaccharide with a hydrazide linker. The hydrazide linker can suitably be adipic acid dihydrazide or hydrazine.

In yet another embodiment of the invention, an aforementioned method is provided further comprising the step of reducing an oxime or hydrazone linkage in the conjugated blinatumomab, for example by incubating the conjugated blinatumomab in a buffer comprising a reducing compound selected from the group consisting of sodium cyanoborohydride (NaCNBH₃) and ascorbic acid (vitamin C). In a related embodiment the reducing compound is sodium cyanoborohydride (NaCNBH₃).

In another embodiment of the invention, a conjugated blinatumomab produced by any aforementioned method is provided. In still another embodiment of the invention, a conjugated blinatumomab; and at least one aminooxy polysaccharide bound to blinatumomab, wherein the aminooxy polysaccharide is attached to blinatumomab, via one or more carbohydrate moieties. In a still further embodiment of the invention, a conjugated blinatumomab; and at least one hydrazide polysaccharide bound to the blinatumomab, wherein the hydrazide polysaccharide is attached to blinatumomab via one or more carbohydrate moieties.

The pharmacological and immunological properties of carbohydrate-containing compounds, such as blinatumomab can be improved by chemical modification and conjugation with polysaccharide, in particular PEG or PSA or mPSA. The properties of the resulting conjugates generally strongly depend on the structure and the size of the polymer. Thus, polymers with a defined and narrow size distribution are usually preferred. PSA and mPSA, used in specific examples, can be purified in such a manner that results in a final PSA preparation with a narrow size distribution.

In yet another embodiment, disclosed herein is a process for producing an aldehyde derivative of a sialic acid compound in which a starting material having a sialic acid unit at its reducing terminal is subjected to sequential steps of
a) reduction to reductively open the ring of the reducing terminal sialic acid unit whereby a vicinal diol group is formed; and
b) selective oxidation to oxidize the vicinal diol group formed in step a) to form an aldehyde group.

The starting material is preferably a di-, oligo- or poly-saccharide although the invention may have utility for other starting materials.

The starting material used in the process preferably will have the sialic acid unit at the reducing terminal end joined to the adjacent unit through its carbon 8 atom. In step b) the 6, 7-diol group is oxidized to form an aldehyde at the carbon 7 atom. In an alternative embodiment, where the sialic acid unit at the reducing terminal end is joined to the adjacent unit through the 9 carbon atom, in step b) a 7, 8 diol group is formed and is oxidized to form an aldehyde on the 8 carbon atom.

In the process of the invention, where the starting material is a di-, oligo- or poly-saccharide, it is preferred that the starting material has a terminal saccharide unit at the non-reducing end which has a vicinal diol group and in which the starting material is subjected to a preliminary step, prior to step a), of selective oxidation to oxidize the vicinal diol group to an aldehyde, whereby in step a) the aldehyde is also reduced to form a hydroxy group which is not part of a vicinal diol group. The invention is of particular utility where the terminal unit of the reducing end of the starting material is a sialic acid unit. In an alternative embodiment the starting material may have a vicinal diol group which is retained as such at a non-reducing terminal saccharide unit of the starting material for step a). It will not be modified by the reduction step but will be oxidized in the oxidation step to form an aldehyde group. The product will be di-functional and may have useful therapeutic activities derived from its ability to cross-link substrates by reaction at both aldehyde groups with suitable functional groups on the substrate.

According to another aspect of the disclosure, there is provided a process in which a sialic acid starting material having a terminal sialic acid at a non-reducing terminal end is subjected to the following steps:
c) a selective oxidation step to oxidize the non-reducing terminal sialic acid unit at the 7, 8 vicinal diol group to form a 7-aldehyde; and
d) a reduction step to reduce the 7-aldehyde group to the corresponding alcohol.

This aspect of the invention provides sialic acid derivatives which have a passivated non-reducing terminal, allowing activation of the reducing terminal for subsequent reaction. The activation may be a reduction/oxidation process e.g. of the first aspect of the invention, with optional subsequent steps of converting the aldehyde group into another group, such as amination to form an amine. Other steps for activating the reducing terminal may be devised.

Preferably this second aspect of the invention is part of a process in which the starting material has a reducing terminal unit and is required to be subsequently conjugated to another molecule through that unit. In such a process the reducing terminal unit is generally activated for instance by a reaction which would otherwise have activated a proportion of the sialic acid non-reducing terminal units were it not for the passivation process. Such a reaction is, for instance selective oxidation of a vicinal diol moiety and is carried out after step d).

It is believed this is the first-time ion-exchange chromatography has been applied to fractionate ionic polysaccharides with molecular weights above about 5 kDa especially polysialic acid of such MWs on the basis of molecular weight. According to a further aspect of this invention there is provided a process for fractionating a population of ionizable polysaccharide with MW higher than 5 kDa using ion-exchange chromatography using in the elution buffer a base or acid which is preferably volatile. Preferably the polysaccharide has carboxylic acid groups and the ion-exchange is anion exchange. Preferably the elution buffer contains an amine, more preferably triethanolamine. Most preferably the polysaccharides are recovered from the fractions by freeze-drying. This method can be applied for the fractionation of CA having other reactive moieties (maleimide or iodoacetate etc.) and other natural (e.g. dextran sulphate) and synthetic (e.g. polyglutamic acid; polylysine in the later case by cation exchange chromatography) charged polymers. It is believed that it is also the first time that IEC has been used to separate ionic polysaccharides in combination with precipitation techniques and/or ultrafiltration methods. The IEC method should remove by-products of production which remain in the commercially available PSAs and CAs, such as endotoxins.

In a preliminary oxidation step and step c) the selective oxidation should preferably be carried out under conditions such that there is substantially no mid-chain cleavage of the the backbone of a long-chain (polymeric) starting material, that is substantially no molecular weight reduction. Enzymes which are capable of carrying out this step may be used. Most conveniently the oxidation is a chemical oxidation. The reaction may be carried out with immobilized reagents such as polymer-based perrhuthenate. The most straight forward method is carried out with dissolved reagents. The oxidant is suitably perrhuthenate, or, preferably, periodate. Oxidation may be carried out with periodate at a concentration in the range 1 mM to 1M, at a pH in the range 3 to 10, a temperature in the range 0 to 60° C. for a time in the range 1 min to 48 hours.

In the process, step a) is a step in which the sialic acid unit at the reducing end is reduced. Usually the unit at the reducing end of the starting material is in the form of a ketal ring and reduction in step a) opens the ring and reduces the ketone to an alcohol. The hydroxyl group at the 6-carbon atom is thus part of a vicinal diol moiety.

Suitable reduction conditions (for steps a) and d)) may utilize hydrogen with catalysts or, preferably hydrides, such as borohydrides. These may be immobilized such as Amberlite (trade mark)-supported borohydride. Preferably alkali metal hydrides such as sodium borohydride is used as the reducing agent, at a concentration in the range 1 μM to 0.1M, a pH in the range 6.5 to 10, a temperature in the range 0 to 60° C. and a period in the range 1 min to 48 hours. The reaction conditions are selected such that pendant carboxyl groups on the starting material are not reduced. Where a preliminary oxidation step has been carried out, the aldehyde group generated is reduced to an alcohol group not part of a vicinal diol group. Other suitable reducing agents are cyanoborohydride under acidic conditions, e.g. polymer supported cyanoborohydride or alkali metal cyanoborohydride, L-ascorbic acid, sodium metabisulphite, L-selectride, triacetoxyborohydride etc.

Between any preliminary oxidation step and reduction step a) and after step b) and between oxidation step c) and reduction step d) and between step d) and any subsequent oxidation step, the respective intermediate must be isolated from oxidizing and reducing agents, respectively, prior to being subjected to the subsequent step. Where the steps are carried out in solution phase, isolation may be by conventional techniques such as expending excess oxidizing agent using ethylene glycol, dialysis of the polysaccharide and ultrafiltration to concentrate the aqueous solution. The product mixture from the reduction step again may be separated by dialysis and ultrafiltration. It may be possible to devise reactions carried out on immobilized oxidizing and reducing reagents rendering isolation of product straightforward.

The selective oxidation step, step b) is suitably carried out under similar conditions to the preliminary oxidation step as described above. Likewise, the oxidation agent should be exhausted before recovery of the product using ethylene glycol. The product is subsequently recovered by suitable means such as dialysis and ultrafiltration.

The process of the first aspect of the invention and of the preferred embodiment of the second aspect which includes a subsequent oxidation step after step d) to activate a reducing terminal saccharide unit produces an activated derivative having a reactive aldehyde moiety derived from the reducing terminal. The preferred process involving an oxidation, then reduction, then oxidation step produces an activated product having a single reactive aldehyde moiety. If there is no preliminary oxidation step and the starting material has a non-reducing terminal unit which has a vicinal diol group (e.g. a sialic acid), the product will have aldehyde groups at each terminal which may have utility.

Aldehyde groups are suitable for conjugating to amine-group containing substrates or hydrazine compounds. Processes in which the activated product of an oxidation step is subsequently conjugated to substrate compound form a further aspect of the invention. Preferably the conjugation reaction is as described in our earlier publications mentioned above, that is involving conjugation with an amine to form a Schiff base, preferably followed by reduction to form a secondary amine moiety. The process is of particular value for derivatizing proteins, of which the amine group is suitably the epsilon amine group of a lysine group or the N-terminal amino group. The process is of particular value for derivatizing protein or peptide therapeutically active agents, such as blinatumomab, cytokines, growth hormones, enzymes, hormones, antibodies or fragments. Alternatively, the process may be used to derivatize drug delivery systems, such as liposomes, for instance by reacting the aldehyde with an amine group of a liposome forming component. Other drug delivery systems are described in our earlier case U.S. Pat. No. 5,846,951. Other materials that may be derivatized include viruses, microbes, cells, including animal cells and synthetic polymers.

Alternatively, the substrate may have a hydrazine group, in which case the product is a hydrazone. This may be reduced if desired, for additional stability, to an alkyl hydrazide.

In another preferred embodiment, oxidation step b) or a subsequent oxidation step after step d) is followed by the reaction of the or each aldehyde group with a linker compound, comprising an amine group or a hydrazide group and another functional group suitable for selective derivatization of proteins or other therapeutically active compounds or drug delivery systems. Such a linker may comprise a compound having a functional group substituent for specific reaction with sulfhydryl groups and a di-basic organic group joining the amine or hydrazide group and the functional group. Reaction of an aldehyde moiety with the amino or hydrazide group forms a reactive conjugate suitable for binding to a substrate having a thiol (sulfhydryl) group. Such conjugates are of particular value for selective and site-directed derivatization of proteins and peptides.

The derivatization of proteins and drug delivery systems may result in increased half-life, improved stability, reduced immunogenicity, and/or control of solubility and hence bioavailability and pharmaco-kinetic properties or may enhance solubility actives or viscosity of solutions containing the derivatized active.

According to the invention there is also provided a compound which is an aldehyde derivative of a di-, oligo or polysaccharide comprising sialic acid moieties, in which the terminal unit at the reducing end is a group OR in which R is selected from —CH₂—CHO,

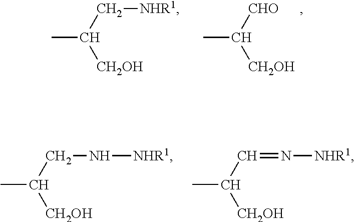

—CH₂CH₂NHR¹, CH₂CH═N—NHR¹ and CH₂CH₂NHNHR¹ in which R¹ is blinatumomab linked through the N terminal.

The novel compound may comprise mid-chain saccharide units between the two terminal units. The mid-chain units may consist only of sialic acid units or, alternatively, may comprise other saccharide units in addition to the terminal units which are derived from sialic acid units. The compound may generally be formed as described above in relation to the first aspect of the invention.

The novel compound may be a polysialylated substrate, comprising at least one polysialic acid (polysaccharide) group conjugated on each molecule of substrate, the conjugation including a secondary amine, hydrazone or alkyl hydrazide linkage via the reducing terminal of the polysialic acid, and is substantially free of crosslinking via the non-reducing end of the polysialic acid group to another molecule of substrate. The substrate may be, for instance, a biologically active compound, for instance a pharmaceutically active compound, especially a peptide or protein therapeutic such as blinatumomab, or a drug delivery system.

The novel compound may have the general formula I

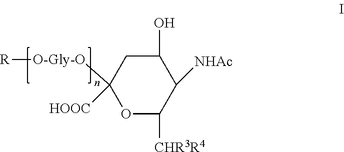

in which R is selected from —CH₂—CHO,

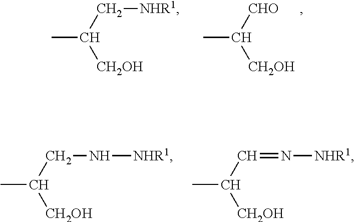

—CH₂CH₂NHR¹, CH₂CH═N—NHR¹ and CH₂CH₂NHNHR¹ in which R¹ is blinatumomab linked through the N terminal;

R³ and R⁴ are selected from
i) R³ is H and R⁴ is OH
ii) where R is CH(CH₂OH)CH₂OH or —CH₂CHO, R³ and R⁴ together are ═O;
iii) where R is CH(CH₂OH)CH₂NHR¹ or —CH₂CH₂NHR¹, R³ is H and R⁴ is —NHR¹;
iv) where R is —CH(CH₂OH)CH₂NHNHR¹ or —CH₂CH₂NHNHR¹, R³ is H and R⁴ is —NHNHR¹; or
v) —CH₂CH═N—NHR¹, R³ and R⁴ are together ═N—NHR¹;
Ac is acetyl
n is 0 or more; and
GlyO is a glycosyl group.

Where R is a group

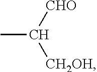

the compound of the general formula I is the polysaccharide which is polysialic acid derivative having an aldehyde group at the reducing terminal unit.

Where R is a group

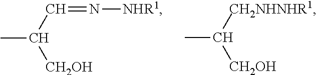

CH₂CH═N—NHR¹ or CH₂CH₂NHNHR¹ the compound is a conjugate formed by reacting the aldehyde derivative of the polysialic acid with a hydrazide R¹NHNH₂. A hydrazide is preferably an acyl hydrazide (R¹ has a terminal carbonyl group).

Where R is a group

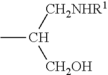

or CH₂CH₂NHR¹, the compound is a conjugate formed by reacting the aldehyde derivative of the polysialic acid with a primary amine group containing compound R¹NH₂.

R¹ is blinatumomab. The group R¹ may further comprise a linker moiety from the active compound to the polysialic acid.

Alternatively, R¹ may be the residue of a linker reagent, for instance to form a derivatized polysialic acid suitable for conjugating to groups other than amine groups or hydrazides on active compounds. Examples are linker reagents of the formula

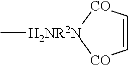

that is a N-maleimido compound, in which R² is a dibasic organic group, for instance an arylene oligo(alkoxy)alkane or, preferably, alkanediyl group, for instance a C₂₋₁₂-alkane diyl group.

The present invention is of most utility where the novel compound is mono-functional and is passivated at the terminal unit at the non-reducing end. In such compounds R³ is H and R⁴ is OH. R can be any of the meanings set out above. The glycosyl groups preferably comprise sialic acid units and more preferably consist only of such units, linked 2-8 and/or 2-9, e.g. alternating 2-8/2-9, to one another.

As used herein “biologically active derivative” or “biologically active variant” includes any derivative or variant of a molecule having substantially the same functional and/or biological properties of the molecule, such as binding properties, and/or the same structural basis, such as a peptide backbone or a basic polymeric unit.

An “analog,” “variant” or “derivative” is a compound substantially similar in structure and having the same biological activity, albeit in certain instances to a differing degree, to a naturally-occurring molecule. For example, a polypeptide variant refers to a polypeptide sharing substantially similar structure and having the same biological activity as a reference polypeptide. Variants or analogs differ in the composition of their amino acid sequences compared to the naturally-occurring polypeptide from which the analog is derived, based on one or more mutations involving (i) deletion of one or more amino acid residues at one or more termini of the polypeptide and/or one or more internal regions of the naturally-occurring polypeptide sequence (e.g., fragments), (ii) insertion or addition of one or more amino acids at one or more termini (typically an “addition” or “fusion”) of the polypeptide and/or one or more internal regions (typically an “insertion”) of the naturally-occurring polypeptide sequence or (iii) substitution of one or more amino acids for other amino acids in the naturally-occurring polypeptide sequence. By way of example, a “derivative” refers to a polypeptide sharing the same or substantially similar structure as a reference polypeptide that has been modified, e.g., chemically.

Variant or analog polypeptides include insertion variants, wherein one or more amino acid residues are added to a protein amino acid sequence of the invention. Insertions may be located at either or both termini of the protein, and/or may be positioned within internal regions of the protein amino acid sequence. Insertion variants, with additional residues at either or both termini, include for example, fusion proteins and proteins including amino acid tags or other amino acid labels. In one aspect, the protein molecule optionally contains an N-terminal Met, especially when the molecule is expressed recombinantly in a bacterial cell such as E. coli.

In deletion variants, one or more amino acid residues in a protein or polypeptide as described herein are removed. Deletions can be effected at one or both termini of the protein or polypeptide, and/or with removal of one or more residues within the protein amino acid sequence. Deletion variants, therefore, include fragments of a protein or polypeptide sequence.

In substitution variants, one or more amino acid residues of a protein or polypeptide are removed and replaced with alternative residues. In one aspect, the substitutions are conservative in nature and conservative substitutions of this type are well known in the art. Alternatively, the invention embraces substitutions that are also non-conservative.

In one embodiment a conjugated compound of the present invention may be administered by injection, such as intravenous, intramuscular, or intraperitoneal injection. The compositions may be useful as therapeutic, diagnostic and/or similar agents.

To administer compositions comprising a conjugated compound of the present invention to human or test animals, in one aspect, the compositions comprise one or more pharmaceutically acceptable carriers. The terms “pharmaceutically” or “pharmacologically acceptable” refer to molecular entities and compositions that are stable, inhibit protein degradation such as aggregation and cleavage products, and in addition do not produce allergic, or other adverse reactions when administered using routes well-known in the art, as described below. “Pharmaceutically acceptable carriers” include any and all clinically useful solvents, dispersion media, coatings, antibacterial and antifungal agents, isotonic and absorption delaying agents and the like, including those agents disclosed above.

As used herein, “effective amount” includes a dose suitable for treating a mammal having a clinically defined disorder.

The compositions may be administered orally, topically, transdermally, parenterally, by inhalation spray, vaginally, rectally, or by intracranial injection. The term parenteral as used herein includes subcutaneous injections, intravenous, intramuscular, intracisternal injection, or infusion techniques. Administration by intravenous, intradermal, intramuscular, intramammary, intraperitoneal, intrathecal, retrobulbar, intrapulmonary injection and or surgical implantation at a particular site is contemplated as well. Generally, compositions are essentially free of pyrogens, as well as other impurities that could be harmful to the recipient.

Single or multiple administrations of the compositions can be carried out with the dose levels and pattern being selected by the treating physician. For the prevention or treatment of disease, the appropriate dosage will depend on the type of disease to be treated, as described above, the severity and course of the disease, whether drug is administered for preventive or therapeutic purposes, previous therapy, the patient's clinical history and response to the drug, and the discretion of the attending physician.

The present invention also relates to a pharmaceutical composition comprising an effective amount of a conjugated compound or protein as defined herein. The pharmaceutical composition may further comprise a pharmaceutically acceptable carrier, diluent, salt, buffer, or excipient. The pharmaceutical composition can be used for treating clinically-defined disorders. The pharmaceutical composition of the invention may be a solution or a lyophilized product. Solutions of the pharmaceutical composition may be subjected to any suitable lyophilization process.

As an additional aspect, the invention includes kits which comprise a composition of the invention packaged in a manner which facilitates its use for administration to subjects. In one embodiment, such a kit includes a compound or composition described herein (e.g., a composition comprising a conjugated protein), packaged in a container such as a sealed bottle or vessel, with a label affixed to the container or included in the package that describes use of the compound or composition in practicing the method. In one embodiment, the kit contains a first container having a composition comprising a conjugated protein and a second container having a physiologically acceptable reconstitution solution for the composition in the first container. In one aspect, the compound or composition is packaged in a unit dosage form. The kit may further include a device suitable for administering the composition according to a specific route of administration. Preferably, the kit contains a label that describes use of the therapeutic protein or peptide composition.

In one embodiment, the derivative retains the full functional activity of native therapeutic compounds, and provides an extended half-life in vivo, as compared to native therapeutic compounds. In another embodiment, the derivative retains at least 20, 21, 22, 23, 24, 25, 26, 27, 28, 29, 30, 31, 32, 34, 35, 36, 37, 38, 39, 40, 41, 42, 43, 44. 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, or 150 percent (%) biological activity relative to native compound.

As used herein, “sialic acid moieties” includes sialic acid monomers or polymers (“polysaccharides”) which are soluble in an aqueous solution or suspension and have little or no negative impact, such as side effects, to mammals upon administration of the PSA-protein conjugate in a pharmaceutically effective amount. PSA and mPSA are characterized, in one aspect, as having 1, 2, 3, 4, 5, 10, 20, 30, 40, 50, 60, 70, 80, 90, 100, 200, 300, 400, or 500 sialic acid units. In certain aspects, different sialic acid units are combined in a chain.

In one embodiment of the invention, the sialic acid portion of the PSA or mPSA compound is highly hydrophilic, and in another embodiment the entire compound is highly hydrophilic. Hydrophilicity is conferred primarily by the pendant carboxyl groups of the sialic acid units, as well as the hydroxyl groups. The saccharide unit may contain other functional groups, such as, amine, hydroxyl or sulphate groups, or combinations thereof. These groups may be present on naturally-occurring saccharide compounds or introduced into derivative polysaccharide compounds. The PSA and mPSA used in the methods and conjugates of the invention may be further characterized as described above in the Background of the Invention.

The naturally occurring polymer PSA is available as a polydisperse preparation showing a broad size distribution (e.g. Sigma C-5762) and high polydispersity (PD). Because the polysaccharides are usually produced in bacteria carrying the inherent risk of copurifying endotoxins, the purification of long sialic acid polymer chains may raise the probability of increased endotoxin content. Short PSA molecules with 1-4 sialic acid units can also be synthetically prepared (Kang S H et al., Chem Commun. 2000; 227-8; Ress D K and Linhardt R J, Current Organic Synthesis. 2004; 1:31-46), thus minimizing the risk of high endotoxin levels. However, PSA preparations with a narrow size distribution and low polydispersity, which are also endotoxin-free, can now be manufactured. Polysaccharide compounds of particular use for the invention are, in one aspect, those produced by bacteria. Some of these naturally-occurring polysaccharides are known as glycolipids. In one embodiment, the polysaccharide compounds are substantially free of terminal galactose units.

In various embodiments, the compound is linked to or associated with the PSA or mPSA compound in stoichiometric amounts (e.g., 1:1, 1:2, 1:3, 1:4, 1:5, 1:6, 1:7, 1:7, 1:8, 1:9, or 1:10, etc.). In various embodiments, 1-6, 7-12 or 13-20 PSA and/or mPSA units are linked to the compound. In still other embodiments, 1, 2, 3, 4, 5, 6, 7, 8, 9, 10, 11, 12, 13, 14, 15, 16, 17, 18, 19, 20 or more PSA and/or mPSA units are linked to the compound.

Optionally, the compound is modified to introduce glycosylation sites (i.e., sites other than the native glycosylation sites). Such modification may be accomplished using standard molecular biological techniques known in the art. Moreover, the compound, prior to conjugation via one or more carbohydrate moieties, may be glycosylated in vivo or in vitro.

In one embodiment of the invention, the reaction of hydroxylamine or hydroxylamine derivatives with aldehydes (e.g., on a carbohydrate moiety following oxidation by sodium periodate) to form an oxime group is applied to the preparation of conjugates of compound. For example, a blinatumomab is first oxidized with a oxidizing agent such as sodium periodate (NalO₄) (Rothfus J A et Smith E L., J Biol Chem 1963, 238, 1402-10; and Van Lenten L and Ashwell G., J Biol Chem 1971, 246, 1889-94). The periodate oxidation of e.g. blinatumomabs is based on the classical Malaprade reaction described in 1928, the oxidation of vicinal diols with periodate to form an active aldehyde group (Malaprade L., Analytical application, Bull Soc Chim France, 1928, 43, 683-96). Additional examples for such an oxidizing agent are lead tetraacetate (Pb(OAc)₄), manganese acetate (MnO(Ac)₃), cobalt acetate (Co(OAc)₂), thallium acetate (TIOAc), cerium sulfate (Ce(SO₄)₂) (U.S. Pat. No. 4,367,309) or potassium perruthenate (KRuO₄) (Marko et al., J Am Chem Soc 1997, 119, 12661-2). By “oxidizing agent” a mild oxidizing compound which is capable of oxidizing vicinal diols in carbohydrates, thereby generating active aldehyde groups under physiological reaction conditions is meant.

The second step is the coupling of the polymer containing an aminooxy group to the oxidized carbohydrate moiety to form an oxime linkage. In one embodiment of the invention, this step can be carried out in the presence of catalytic amounts of the nucleophilic catalyst aniline or aniline derivatives (Dirksen A et Dawson P E, Bioconjugate Chem. 2008; Zeng Y et al., Nature Methods 2009; 6:207-9). The aniline catalysis dramatically accelerates the oxime ligation allowing the use of very low concentrations of the reagents. In another embodiment of the invention the oxime linkage is stabilized by reduction with NaCNBH₃ to form an alkoxyamine linkage.

In one embodiment of the invention, the reaction steps to conjugate PSA or mPSA to a protein are carried out separately and sequentially (i.e., starting materials (e.g., protein, polymer, etc), reagents (e.g., oxidizing agents, aniline, etc) and reaction products (e.g., oxidized carbohydrate on a protein, activated aminooxy polymer, etc) are separated between individual reaction steps).

Additional information on aminooxy technology can be found in the following references, each of which is incorporated in their entireties: EP 1681303A1 (HASylated erythropoietin); WO 2005/014024 (conjugates of a polymer and a protein linked by an oxime linking group); WO96/40662 (aminooxy-containing linker compounds and their application in conjugates); WO 2008/025856 (Modified proteins); Peri F et al., Tetrahedron 1998, 54, 12269-78; Kubler-Kielb J and Pozsgay V., J Org Chem 2005, 70, 6887-90; Lees A et al., Vaccine 2006, 24(6), 716-29; and Heredia K L et al., Macromoecules 2007, 40(14), 4772-9.

Advantages of the invention include high recovery of conjugate, high retention of activity of the conjugated blinatumomab as compared to the unconjugated form and high conjugation efficiency.

### EXAMPLES

The following non-limiting examples are provided for illustrative purposes only in order to facilitate a more complete understanding of representative embodiments now contemplated. These examples are intended to be a mere subset of all possible contexts in which the composition may be utilized. Thus, these examples should not be construed to limit any of the embodiments described in the present specification, including those pertaining to compositions and/or methods and uses thereof. Ultimately, the composition may be utilized in virtually any context where the treatment and/or control of pain is desired.

### Example 1

### Coupling of Aminooxy-PSA to Blinatumomab and Purification of the Conjugate

To 12.6 mg blinatumomab, dissolved in 6.3 ml 50 mM sodium acetate buffer, pH 6.0, 289 μl of an aqueous sodium periodate solution (10 mM) was added. The mixture was shaken in the dark for 1 h at 4° C. and quenched for 15 min at room temperature by the addition of 6.5 μl 1M glycerol. Low molecular weight contaminates were removed by ultrafiltration/diafiltration (UF/DF) employing Vivaspin (Sartorius, Goettingen, Germany) concentrators (30 kD membrane, regenerated cellulose). Next, 43 mg aminooxy-PSA was added to the UF/DF retentate and the mixture was shaken for 18 hrs at 4° C. The excess PSA reagent was removed by hydrophobic interaction chromatography (HIC). The conductivity of the cooled reaction mixture was raised to 180 mS/cm and loaded onto a 5 ml HiTrap Butyl FF (GE Healthcare, Fairfield, Conn.) HIC column (1.6×2.5 cm), pre-equilibrated with 50 mM HEPES, 3M sodium chloride, 6.7 mM calcium chloride, 0.01% Tween 80, pH 6.9. The conjugate was eluted within 2.4 column volumes (CV) with 50 mM HEPES, 6.7 mM calcium chloride, 0.005% Tween 80, pH 7.4 at a flow rate of 5 ml/min. The preparation was analytically characterized by measuring total protein (BCA) and FIX chromogenic activity. For the PSA-blinatumomab conjugate a specific activity of 80.2 IU/mg protein was determined (56.4% in comparison to native blinatumomab).

### Example 2

### Coupling of Aminooxy-PSA to Blinatumomab in the Presence of Aniline as Nucleophilic Catalyst

To 3.0 mg blinatumomab, dissolved in 1.4 ml 50 mM sodium acetate buffer, pH 6.0, 14.1 μl of an aqueous sodium periodate solution (10 mM) was added. The mixture was shaken in the dark for 1 h at 4° C. and quenched for 15 min at room temperature by the addition of 1.5 μl 1 M glycerol. Low molecular weight contaminates were removed by means of size exclusion chromatography (SEC) employing PD-10 desalting columns (GE Healthcare, Fairfield, Conn.). 1.2 mg oxidized blinatumomab, dissolved in 1.33 ml 50 mM sodium acetate buffer, pH 6.0 was mixed with 70 μl of aniline (200 mM aqueous stock solution) and shaken for 45 min at room temperature. Next, 4.0 mg aminooxy-PSA was added and the mixture was shaken for 2 hrs at room temperature and another 16 hrs at 4° C. Samples were drawn after 1 h, after 2 hrs and at the end of the reaction after 18 hrs. Next, excess PSA reagent and free blinatumomab were removed by means of HIC. The conductivity of the cooled reaction mixture was raised to 180 mS/cm and loaded onto a 5 ml HiTrap Butyl FF (GE Healthcare, Fairfield, Conn.) HIC column (1.6×2.5 cm), pre-equilibrated with 50 mM HEPES, 3M sodium chloride, 6.7 mM calcium chloride, 0.01% Tween 80, pH 6.9. The conjugate was eluted with a linear gradient to 50 mM HEPES, 6.7 mM calcium chloride, 0.005% Tween 80, pH 7.4 in 20 CV with at a flow rate of 5 ml/min.

### Example 3

### Coupling of Aminooxy-PSA to Blinatumomab and Reduction with NaCNBH₃

To 10.5 mg blinatumomab, dissolved in 5.25 ml 50 mM sodium acetate buffer, pH 6.0, 53 μl of an aqueous sodium periodate solution (10 mM) was added. The mixture was shaken in the dark for 1 h at 4° C. and quenched for 15 min at room temperature by the addition of 5.3 μl 1 M glycerol. Low molecular weight contaminates were removed by means of UF/DF employing Vivaspin (Sartorius, Goettingen, Germany) concentrators (30 kD membrane, regenerated cellulose). Next, 35.9 mg aminooxy-PSA was added to the UF/DF retentate and the mixture was shaken for 2 hrs at room temperature. Then 53 μl of aqueous sodium cyanoborohydride solution (5M) was added and the reaction was allowed to proceed for another 16 hrs. Then the excess PSA reagent was removed by means of HIC. The conductivity of the cooled reaction mixture was raised to 180 mS/cm and loaded onto a 5 ml HiTrap Butyl FF HIC (GE Healthcare, Fairfield, Conn.) column (1.6×2.5 cm), pre-equilibrated with 50 mM HEPES, 3M sodium chloride, 6.7 mM calcium chloride, 0.01% Tween 80, pH 6.9. The conjugate was eluted within 2.4 CV with 50 mM HEPES, 6.7 mM calcium chloride, 0.005% Tween 80, pH 7.4 at a flow rate of 5 ml/min.

### Example 4

### Coupling of Aminooxy-PSA (Linker: NH₂[OCH₂CH₂]₄ONH₂) to Blinatumomab and Purification of the Conjugate

To 5.6 mg blinatumomab, dissolved in 2.8 ml 50 mM sodium acetate buffer, pH 6.0, 102 μl of an aqueous solution of sodium periodate (10 mM) was added. The mixture was shaken in the dark for 1 h at 4° C. and quenched for 15 min at room temperature by the addition of 2.9 μl of 1M glycerol. Low molecular weight contaminates were removed by means of UF/DF employing Vivaspin (Sartorius, Goettingen, Germany) concentrators (30 kD membrane, regenerated cellulose). Then 19 mg aminooxy-PSA was added to the UF/DF retentate and the mixture was shaken for 18 hrs at 4° C. The excess PSA reagent was removed by means of HIC. The conductivity of the cooled reaction mixture was raised to 180 mS/cm and loaded onto a 5 ml HiTrap Butyl FF (GE Healthcare, Fairfield, Conn.) HIC column (1.6×2.5 cm), pre-equilibrated with 50 mM HEPES, 3M sodium chloride, 6.7 mM calcium chloride, 0.01% Tween 80, pH 6.9. The conjugate was eluted within 2.4 CV with 50 mM HEPES, 6.7 mM calcium chloride, 0.005% Tween 80, pH 7.4 at a flow rate of 5 ml/min.

### Example 5

### Polysialylation of Blinatumomab Employing a Maleimido/Aminooxy Linker System

### A. Preparation of the Modification Reagent

An Aminooxy-PSA reagent is prepared by use of a maleimido/aminooxy linker system (Toyokuni et al., Bioconjugate Chem 2003; 14, 1253-9). PSA-SH (20 kD) containing a free terminal SH—group is prepared using a two-step procedure: a) Preparation of PSA-NH₂ by reductive amination of oxidized PSA with NH₄Cl according to WO05016973A1 and b) introduction of a sulfhydryl group by reaction of the terminal primary amino group with 2-iminothiolane (Traut's reagent/Pierce, Rockford, Ill.) as described in U.S. Pat. No. 7,645,860. PSA-SH is coupled to the maleimido-group of the linker at pH 7.5 in PBS—buffer using a 10-fold molar excess of the linker and a PSA-SH concentration of 50 mg/ml. The reaction mixture is incubated for 2 hours under gentle shaking at room temperature. Then the excess linker reagent is removed, and the aminooxy-PSA is buffer exchanged into oxidation buffer (50 mM sodium phosphate, pH 6.0) by diafiltration. The buffer is exchanged 25 times employing a Pellicon XL5 kD regenerated cellulose membrane (Millipore, Billerica, Mass.).

B. Modification of Blinatumomab after Prior Oxidation with NalO₄

Blinatumomab is oxidized in 50 mM sodium phosphate buffer, pH 6.0 employing 100 μM sodium periodate in the buffer. The mixture was shaken in the dark for 1 h at 4° C. and quenched for 15 min at room temperature by the addition of glycerol to a final concentration of 5 mM. Low molecular weight contaminates were removed by means of size exclusion chromatography (SEC) employing PD-10 desalting columns (GE Healthcare, Fairfield, Conn.). Oxidized blinatumomab is then spiked with aniline to obtain a final concentration of 10 mM and mixed with the aminooxy-PSA reagent to achieve a 5-fold molar excess of PSA. The reaction mixture was incubated for 2 hours under gentle shaking in the dark at room temperature.

### C. Purification of the Conjugates

The excess of PSA reagent and free blinatumomab is removed by means of HIC. The conductivity of the reaction mixture is raised to 180 mS/cm and loaded onto a column filled with 48 ml Butyl-Sepharose FF (GE Healthcare, Fairfield, Conn.) pre-equilibrated with 50 mM Hepes, 3 M sodium chloride, 6.7 mM calcium chloride, 0.01% Tween 80, pH 6.9. Subsequently the conjugate is eluted with a linear gradient of 60% elution buffer (50 mM Hepes, 6.7 mM calcium chloride, pH 7.4) in 40 CV. Finally, the PSA-blinatumomab containing fractions are collected and subjected to UF/DF by use of a 30 kD membrane made of regenerated cellulose (Millipore). The preparation is analytically characterized by measuring total protein (BCA) and FIX chromogenic activity. For the PSA-blinatumomab conjugates prepared with both variants a specific activity of >50% in comparison to native blinatumomab was determined.

### Example 6

### Coupling of Diaminooxy (3,6,9-trioxa-undecane-1,11-dioxyamine)—PSA to Blinatumomab

1.9 mg of blinatumomab was oxidized with 1.5 mM of NalO₄ for 30 minutes at 4° C. and then oxidation was stopped by adding NaHSO₃ to a final concentration of 5 mM. The conjugation reaction was carried out using the oxidized blinatumomab with diaminooxy PSA polymer. The final concentrations of polymer and protein in the reaction mixture were 1.25 mM and 0.76 mg/ml respectively. The final pH of the reaction mixture was around 5.75. Sodium cyanoborohydride was added to the reaction mixture to a concentration of 50 mM or 3.17 mg/ml. The reaction was carried out at 4° C. for 2 hours. Purified and unpurified conjugates were characterized using SDS PAGE and western blotting. A shift in the band was seen for the conjugate in SDS PAGE and this was confirmed by western blotting using anti-PSA antibody. The in vitro activity PSA-blinatumomab conjugates were comparable to native protein using All in one βGal assay kit (Pierce). Less than 50% activity was observed in comparable conjugates made using aldehyde linker chemistry. Further, the overall process was scaled up to 3 fold.

### Example 7

### Coupling of Diaminooxy-PSA to Blinatumomab

5 mg of blinatumomab was oxidized with 10 mM NalO₄ for 60 minutes at 4° C. in dark and then oxidation was stopped by adding NaHSO₃ to a final concentration of 10 mM. The conjugation reaction was carried out using the oxidized blinatumomab with diaminooxy PSA polymer (23 kDa). The final concentration of polymer in the reaction mixture was 2.5 mM at pH of 5.75. Sodium cyanoborohydride was added to the reaction mixture to a concentration of 50 mM or 3.17 mg/ml. The reaction was carried out at 4° C. and sample was collected after 2 hours. Purified and unpurified conjugates were characterized using SDS PAGE and western blotting. A shift in the band was seen for the conjugate in SDS PAGE and this was also confirmed by western blotting.

### Example 8

### Coupling of Diaminooxy-PSA to Blinatumomab with Aniline to Act as a Nucleophilic Catalyst

0.2 mg of blinatumomab was oxidized with 10 mM NalO₄ for 30 minutes at 4° C. in dark and then oxidation was stopped by adding NaHSO₃ to a final concentration of 5 mM. The conjugation reaction was carried out using the oxidized blinatumomab with diaminooxy PSA polymer (23 kDa). The final concentration of polymer in the reaction mixture was 1.25 mM. The final pH of reaction mixture was 5.75. Sodium cyanoborohydride was added to the reaction mixture to a concentration of 50 mM or 3.17 mg/ml. The final protein concentration in the reaction was 0.125 mg/ml. 84.21 μl of 200 mM aniline solution was added to the 1.6 ml of reaction mixture. The reaction was carried out at 4° C. overnight.

### Example 9

### Coupling of Diaminooxy (3 Oxa-Pentane-1,5-Dioxyamine Linker)-PSA to Blinatumomab

For oxidation of blinatumomab, NalO₄ was used at a concentration of 2 mM. 3 mg of blinatumomab was oxidized at acidic pH of 5.75 at 4° C. for 30 minutes then oxidation was stopped by adding NaHSO₃ to a final concentration of 2 mM. The conjugation reaction was carried out using the oxidized blinatumomab with diaminooxy PSA polymer (23 kDa). The final concentration of polymer in the reaction mixture was 1.5 mM. The final concentration of blinatumomab in reaction mixture was 0.867 mg/ml. The final pH of reaction mixture was around 5.75. Sodium cyanoborohydride was added to the reaction mixture to a concentration of 50 mM or 3.17 mg/ml. The reaction was carried out at 4° C. for 2 hours. Conjugates were characterized using SDS PAGE and western blotting. A shift in the band was seen for the conjugate in SDS PAGE and positive result was obtained from western blotting.

### Example 10

### Coupling of Hydrazide-PSA to Blinatumomab

For oxidation of blinatumomab, NalO₄ was used at a concentration of 10 mM. blinatumomab (1 mg) was oxidized at pH 5.75 at 4° C. for 30 minutes then oxidation was stopped by adding NaHSO₃ to a final concentration of 5 mM. The conjugation reaction was carried out using the oxidized blinatumomab with hydrazide-PSA polymer. The molecular weight of the hydrazide-PSA used for conjugation was 24.34 kDa. The final concentration of hydrazide-PSA in the reaction mixture was 1.25 mM. The final concentration of EPO in the reaction mixture was 0.125 mg/ml. The final pH of the reaction mixture was around 5.75. Sodium cyanoborohydride was added to the reaction mixture to a concentration of 50 mM or 3.17 mg/ml. The reaction was carried out at 4° C. for 24 hours. Conjugates were characterized using SDS PAGE and western blotting. A shift in the band was seen for the conjugate in SDS PAGE and a positive result was obtained from western blotting.

### Example 11

### Treatment of 32 Year Old Male Suffering from B-Cell Precursor Acute Lymphoblastic Leukemia (ALL) in Remission but Still has Minimal Residual Disease (MRD)

The patient, a 32 year old male is diagnosed by his doctor after claiming he is suffering from weakness and inflamed lymph nodes and a painful pancreas. The patient's doctor diagnoses the patient as suffering from ALL. The patient is put on PSA-blinatumomab therapy and shows a reduction in the symptoms he suffers from ALL (i.e., inflamed lymph nodes and a inflammed pancreas). Three months after administration, the patient's pancreas is found to be normal, with the result that the histone related symptoms the patient suffered from are further reduced and the progression of ALL slows down considerably.

### Example 12

### Treatment of 32 Year Old Male Suffering from Philadelphia Chromosome (pH)-Negative Relapsed or Refractory Positive B-Cell Precursor ALL

The patient, a 45 year old female is diagnosed by her doctor after claiming she is suffering from weakness and inflamed lymph nodes and inflamed liver. The patient's doctor diagnoses the patient as suffering from relapsed ALL. The patient is put on PSA-blinatumomab therapy and shows a reduction in the symptoms she suffers from as related to histones (i.e., inflamed lymph nodes and painful liver). Three months after administration, the patient's liver is found to be normal, with the result that the histone related symptoms the patient suffered from are further reduced and the progression of ALL slows down considerably.

### Example 13

### Treatment of 32 Year Old Male Suffering from Philadelphia Chromosome (pH)-Positive Relapsed or Refractory Positive B-Cell Precursor ALL

The patient, a 52 year old male is diagnosed by his doctor after claiming he is suffering from weakness and inflamed lymph nodes. The patient's doctor diagnoses the patient as suffering from relapsed ALL. The patient is put on PSA-blinatumomab therapy and shows a reduction in the symptoms he suffers from as related to histones (i.e., inflamed lymph nodes and peritonitis). Three months after administration, the patient's histone related symptoms the patient suffered from are further reduced and the progression of ALL slows down considerably.

In closing, regarding the exemplary embodiments of the present disclosure as shown and described herein, it will be appreciated that an inhalation formulation is disclosed and configured for methods of treating pain via inhalation. Because the principles of the disclosure may be practiced in a number of configurations beyond those shown and described, it is to be understood that the disclosure is not in any way limited by the exemplary embodiments but is generally directed to an inhalation formulation and methods of use for treating pain and is able to take numerous forms to do so without departing from the spirit and scope of the disclosure. It will also be appreciated by those skilled in the art that the present disclosure is not limited to the particular geometries and materials of construction disclosed but may instead entail other functionally comparable structures or materials, now known or later developed, without departing from the spirit and scope of the disclosure.

Certain embodiments of the present disclosure are described herein, including the best mode known to the inventor(s) for carrying out the disclosure. Of course, variations on these described embodiments will become apparent to those of ordinary skill in the art upon reading the foregoing description. The inventor(s) expect skilled artisans to employ such variations as appropriate, and the inventor(s) intend for the present disclosure to be practiced otherwise than specifically described herein. Accordingly, this disclosure includes all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein. Similarly, as used herein, unless indicated to the contrary, the term “substantially” is a term of degree intended to indicate an approximation of the characteristic, item, quantity, parameter, property, or term so qualified, encompassing a range that can be understood and construed by those of ordinary skill in the art.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

The terms “a,” “an,” “the” and similar references used in the context of describing the present disclosure (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present disclosure and does not pose a limitation on the scope of the disclosure otherwise claimed. No language in the present specification should be construed as indicating any non-claimed element essential to the practice of the disclosure.

When used in the claims, whether as filed or added per amendment, the open-ended transitional term “comprising” (along with equivalent open-ended transitional phrases thereof such as “including,” “containing” and “having”) encompasses all the expressly recited elements, limitations, steps and/or features alone or in combination with un-recited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amendment for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim, whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (along with equivalent open-ended transitional phrases thereof) includes within its meaning, as a limiting case, claimed subject matter specified by the closed-ended transitional phrases “consisting of” or “consisting essentially of.” As such, embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present disclosure. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents is based on the information available to the applicants and does not constitute any admission as to the correctness of the dates or contents of these documents.

While aspects of the disclosure have been described with reference to at least one exemplary embodiment, it is to be clearly understood by those skilled in the art that the disclosure is not limited thereto. Rather, the scope of the disclosure is to be interpreted only in conjunction with the appended claims and it is made clear, here, that the inventor(s) believe that the claimed subject matter is the disclosure.

1. A composition comprising a population of polysaccharide-blinatumomab conjugates, wherein the polysaccharide is covalently linked to the blinatumomab.

2. The composition of claim 1, wherein the polysaccharide comprises between 2 and 125 saccharide units.

3. The composition of claim 1, wherein the polysaccharide is polysialic acid (PSA), heparin, hyaluronic acid or chondroitin sulphate.

4. The composition of claim 3, wherein the polysaccharide is PSA consisting substantially only of sialic acid units.

5. The composition of claim 1, wherein the blinatumomab is derivatized by the polysaccharide at the reducing terminal unit of the polysaccharide.

6. The composition of claim 1, wherein the polysaccharide derivatives are compounds of Formula (I): 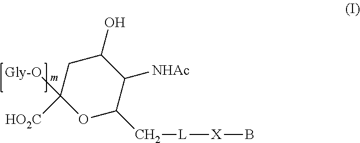
wherein
m is at least one;
XB is derived from B—XH which is blinatumomab, wherein XH is NH₂ or SH;
L is a bond, a linking group, or comprises a polypeptide or a synthetic oligomer;
GlyO is an anionic saccharide unit, and
R² is H or C₁₋₆ alkyl,
wherein the linking group, if present, is of general formula —Y—C(O)—R¹—C(O)—, and
wherein Y is NR² or NR²—NR²; R¹ is a difunctional organic radical selected from the group consisting of alkanediyl, arylene, alkarylene, heteroarylene and alkylheteroarylene, any of which may substituted and/or interrupted by carbonyl, ester, sulfide, ether, amide and/or amine linkages.

7. The composition of claim 6, wherein L is a bond or is a group

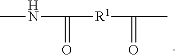

8. The composition of claim 1, wherein the polysaccharides comprise 10-80 sialic acid units.

9. The composition of claim 1, wherein the polydispersity of the anionic polysaccharide is less than 1.3.

10. A composition comprising a population of polysaccharides and blinatumomab.

11. The composition of claim 10, wherein the polysaccharide comprises between 2 and 125 saccharide units.

12. The composition of claim 10, wherein the polysaccharide is polysialic acid (PSA), heparin, hyaluronic acid or chondroitin sulphate.

13. The composition of claim 12, wherein the polysaccharide is PSA consisting substantially only of sialic acid units.

14. The composition of claim 10, wherein the blinatumomab is derivatized by the polysaccharide at the reducing terminal unit of the polysaccharide.

15. The composition of claim 10, wherein the polysaccharides comprise 10-80 sialic acid units.

16. The composition of claim 10, wherein the polydispersity of the anionic polysaccharide is less than 1.3.

17. The composition of claim 1, wherein the composition is a pharmaceutical composition and comprises one or more pharmaceutically acceptable excipients.

18. The composition of claim 10, wherein the composition is a pharmaceutical composition and comprises one or more pharmaceutically acceptable excipients.
